(19) United States
(12) Patent Application Publication
(10) Pub. No.: US 2014/0323846 A1
(43) Pub. Date: Oct. 30, 2014

(54) SYSTEM AND METHOD FOR DETERMINING HEMODYNAMIC STATUS THROUGH A BLOOD PRESSURE RELATED INDEX

(71) Applicant: COVIDIEN LP, Boulder, CO (US)

(72) Inventors: Kathleen H. Niebel, Westminster, MA (US); James Nicholas Watson, Dunfermline (GB); Paul Stanley Addison, Edinburgh (GB)

(73) Assignee: COVIDIEN LP, Boulder, CO (US)

(21) Appl. No.: 14/227,594

(22) Filed: Mar. 27, 2014

Related U.S. Application Data
(60) Provisional application No. 61/815,407, filed on Apr. 24, 2013.

(51) Int. Cl. A61B 5/021; A61B 5/00

(52) U.S. Cl. CPC A61B 5/021; A61B 5/0059
USPC 600/407

(57) ABSTRACT

A system for determining a hemodynamic status of an individual may include a photoplethysmography (PPG) sub-system configured to detect a PPG signal and a response triggering module configured to analyze the PPG signal and output one or more response triggers based on a changing feature of the PPG signal within a time window. Each of the one or more response triggers may relate to an instruction to initiate detection of at least one physiological characteristic of the individual. A blood pressure (BP) variability index determination module is configured to determine a BP variability index related to a hemodynamic status of the individual based on a frequency or pattern of the one or more response triggers.

### CROSS-REFERENCE TO RELATED APPLICATIONS

The present application relates to and claims priority benefits from U.S. Provisional Patent Application No. 61/815,407, entitled “System and Method for Determining Health Status Through a Blood Pressure Variability Index,” filed Apr. 24, 2013, which is hereby expressly incorporated by reference in its entirety.

### FIELD OF THE DISCLOSURE

Embodiments of the present disclosure generally relate to physiological signal processing and, more particularly, to processing physiological signals to determine a hemodynamic status of an individual through a blood pressure related index.

### BACKGROUND OF THE DISCLOSURE

Blood pressure represents a measurement that quantifies a pressure exerted by circulating blood upon walls of blood vessels. In general, blood pressure is an example of a principal vital sign. Typically, blood pressure may be measured through use of a sphygmomanometer, or blood pressure cuff. Blood pressure may also be invasively detected through an arterial line catheter, for example. However, continuous non-invasive blood pressure (CNIBP) monitoring systems are configured to continuously track blood pressure, unlike standard occlusion cuff techniques, and without the hazards of invasive arterial lines.

It has been found that increased levels of blood pressure variability may be associated with subsequent patient complications and cardiovascular events. If a high degree of blood pressure variability can be detected early enough, adverse patient outcomes and complications may be avoided.

### SUMMARY OF EMBODIMENTS OF THE PRESENT DISCLOSURE

Certain embodiments of the present disclosure provide a system for determining a hemodynamic status of an individual. In at least one embodiment, the system includes a physiological monitor, such as a pulse oximeter, that detects a signal representing arterial blood characteristics. This arterial blood signal is analyzed for variations that indicate that the patient's blood pressure may have changed. Based on the analysis, the system determines whether to trigger a new blood pressure measurement, such as by triggering activation of a blood pressure cuff. However, the system may operate without actually taking such a measurement, and instead may simply keep track of how many times the system would have triggered such a measurement. The system tracks the frequency of the triggers over time, regardless of whether a new blood pressure measurement is actually taken. The frequency of the triggers can be used to develop an index or measure of blood pressure variability, which can be displayed and/or used as an input for other algorithms, treatments, alarms, or clinical decisions.

In at least one embodiment, the system may include a photoplethysmography (PPG) sub-system configured to detect a PPG signal, and a response triggering module configured to analyze the PPG signal and output one or more response triggers based on a changing feature of the PPG signal within a time window. Each response trigger may relate to an instruction to initiate detection of at least one physiological characteristic, such as blood pressure, of the individual. A blood pressure (BP) variability index determination module is configured to determine a BP variability index related to a hemodynamic status of the individual based on a frequency or pattern of the response trigger(s). For example, in at least one embodiment, the BP variability index includes a number of the response trigger(s) output by the response triggering module during the time window.

The system may also include a BP detection unit operatively connected to the response trigger module and the BP variability index determination module. The BP detection unit is configured to initiate detection of BP of the individual upon reception of the one or more response triggers. In at least one embodiment, the BP variability index is further based on detected BP of the individual. Alternatively, the system may be devoid of a separate and distinct device configured to detect the physiological characteristic(s) of the individual. For example, the system may operate without a blood pressure cuff or other blood pressure sensor.

The system may also include a fluid responsiveness determination module in communication with the PPG sub-system and the BP variability index determination module. The fluid responsiveness determination module is configured to determine a fluid responsiveness predictor (FRP) based on an analysis of the PPG signal. In at least one embodiment, the BP variability index is further based on the FRP. In at least one embodiment, the one or more response triggers may be based on, or relate to, one or more thresholds of the FRP.

Certain embodiments of the present disclosure provide a method for determining a hemodynamic status of an individual. The method may include detecting a photoplethysmography (PPG) signal with a PPG sub-system, using a response triggering module to analyze the PPG signal, and output one or more response triggers based on changing features of the PPG signal within a time window. Each of the response trigger(s) may relate to an instruction to initiate detection of at least one physiological characteristic of the individual. The method may also include determining a blood pressure (BP) variability index related to a hemodynamic status of the individual based on a frequency or pattern of the response trigger(s).

Certain embodiments of the present disclosure provide a tangible and non-transitory computer readable medium that includes one or more sets of instructions configured to direct a computer to analyze a photoplethysmography (PPG) signal and output one or more response triggers based on changing features of the PPG signal within a time window (in which each of the response triggers may relate to an instruction to initiate detection of at least one physiological characteristic of the individual), and determine a blood pressure (BP) variability index related to a hemodynamic status of the individual based on at least the response trigger(s).

Certain embodiments of the present disclosure relate to a system for determining a hemodynamic status of an individual that may include at least one circuit or processor configured to determine whether to trigger a blood pressure (BP) measurement of the individual based on an analysis of a photoplethysmography (PPG) signal. The circuit(s) and/or processor(s) is also configured to develop a BP variability index related to the hemodynamic status of the individual based on a number of triggered BP measurements within a time window. In at least one embodiment, the system may be devoid of a separate and distinct device configured to detect the BP of the individual.

Certain embodiments of the present disclosure provide a system for determining a hemodynamic status of an individual that may include an input receiving a photoplethysmography (PPG) signal representing light absorption by a subject's tissue, a trigger generated based on a change in a feature of the PPG signal, wherein the trigger indicates a probability of a change in blood pressure of the subject based on the change in the feature of the PPG signal, and a calculator outputting a blood pressure (BP) variability index based on a number or pattern of triggers generated over a period of time.

### BRIEF DESCRIPTION OF THE DRAWINGS

FIG. 1 illustrates a simplified block diagram of a system for determining a hemodynamic status of an individual, according to an embodiment of the present disclosure.

FIG. 2 illustrates a simplified block diagram of a system for determining a hemodynamic status of an individual, according to an embodiment of the present disclosure.

FIG. 3 illustrates a simplified block diagram of a system for determining a hemodynamic status of an individual, according to an embodiment of the present disclosure.

FIG. 4 illustrates a representation of a PPG signal, according to an embodiment of the present disclosure.

FIG. 5 illustrates a chart of a fluid responsiveness parameter over time, according to an embodiment of the present disclosure.

FIG. 6 illustrates a flow chart of a method for determining a hemodynamic status of an individual, according to an embodiment of the present disclosure.

FIG. 7 illustrates a perspective view of a monitoring system, according to an embodiment of the present disclosure.

FIG. 8 illustrates a block diagram of a monitoring system, according to an embodiment of the present disclosure.

Before the embodiments of the disclosure are explained in detail, it is to be understood that the disclosure is not limited in its application to the details of construction and the arrangement of the components set forth in the following description or illustrated in the drawings. The disclosure is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION OF THE DRAWINGS

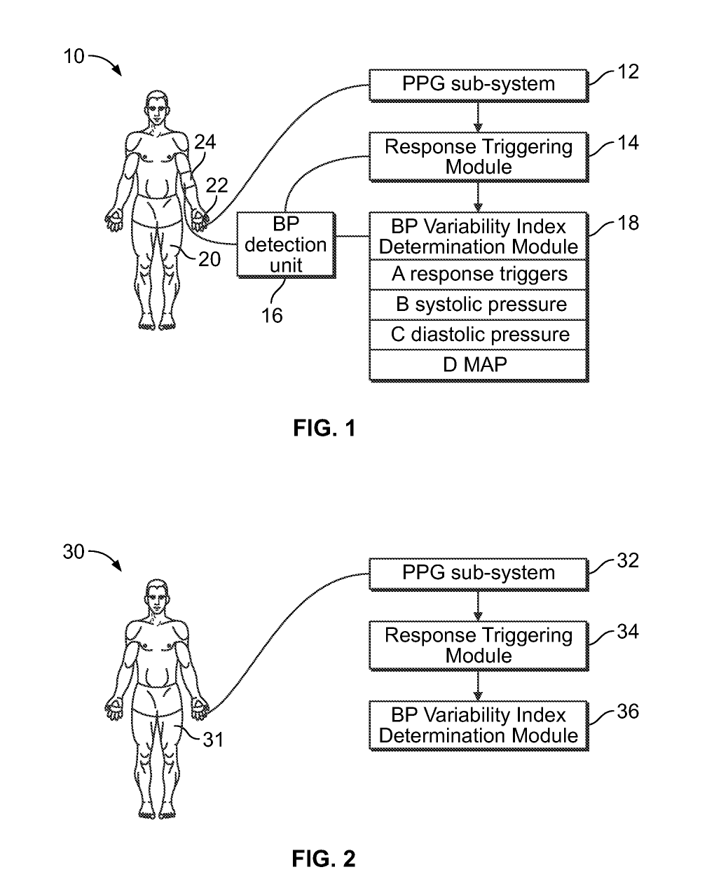

FIG. 1 illustrates a simplified block diagram of a system 10 for determining a hemodynamic status of an individual 20, according to an embodiment of the present disclosure. The system 10 may include a photoplethysmography (PPG) sub-system 12 in communication with a response triggering module 14 that is in communication with a blood pressure (BP) detection unit 16 and a BP variability index determination module or calculator 18.

The PPG sub-system 12 is configured to be operatively connected to the individual 20 and used to detect and output a PPG signal, which is indicative of one or more physiological characteristics of the individual 20. For example, the PPG sub-system 12 may include an input that receives a PPG signal from a PPG sensor 22, such as a pulse oximetry sensor, that may be positioned on a finger, forehead, forearm, or the like of the individual 20. The PPG sensor 22 is configured to detect the PPG signal, which may be in the form of a PPG waveform responsive to the blood flow of the individual. The PPG signal is a non-invasive, optical measurement that may be used to detect changes in blood volume within tissue, such as skin, of an individual. In general, the PPG signal is a physiological signal that includes an AC physiological component related to cardiac synchronous changes in the blood volume with each heartbeat. The AC component may be superimposed on a DC baseline that may be related to respiration, sympathetic nervous system activity, and thermoregulation. The PPG signal may be analyzed to determine physiological characteristics such as respiration rate, respiratory effort, pulse rate, oxygen saturation, and/or the like.

After detecting the PPG signal, the PPG sub-system 12 transmits or otherwise sends the PPG signal to the response triggering module 14. The response triggering module 14 analyzes the PPG signal to determine whether or not to output a response trigger, such as a BP detection trigger, response, output, or the like. For example, when the characteristics or features of the PPG signal (such as an amplitude or frequency of an AC or DC component of the PPG signal) change significantly (such as with respect to one or more thresholds), it may be inferred that the blood pressure is changing. Accordingly, the response triggering module 14 outputs a response trigger, such as the BP detection trigger, which indicates that the BP has likely changed, and a new BP measurement of the individual 20 should be taken. The degree of BP variability may be determined by the number or pattern of response triggers over time. For example, as the frequency of response triggers increases, so too does BP variability (for example, the degree of variability of BP). Hemodynamic status of the individual 20 may be determined based on the degree of BP variability, which may be determined, at least in part, by the number and nature of the response triggers within a time window.

Further, if no response triggers are output, the system 10 may indicate that the blood pressure of the individual 20 is stable. That is, zero response triggers during a relevant time window indicates a stable blood pressure, which may be displayed or otherwise communicated to the individual 20 and/or a clinician.

Each response trigger may be configured to initiate detection or measurement of a physiological characteristic of the individual 20. The physiological characteristic may be a cardiac-related characteristic or parameter, such as blood pressure, pulse rate, cardiac output, and/or the like. For example, a response trigger may include a BP detection response that is output by the response triggering module 14 and configured to initiate BP detection, such as through activation of a BP detection sensor 24 (for example, a BP cuff), which may be secured to the individual 20 and in communication with the BP detection unit 16. Each response trigger is output by the response triggering module 14 after an analysis of the PPG signal indicates that BP should be measured, based on changing characteristics or features of the PPG signal. It is to be noted, however, that the response triggering module 14 may determine and output the response triggers even if the BP detection unit 16 and the BP detection sensor 24 are not operatively connected to the system 10. Indeed, the response triggering module 14 is configured to analyze the PPG signals and output response triggers based on analysis of the PPG signals even when the system 10 is devoid of the BP detection unit 16, or other such sensor. Alternatively, even when a BP detection unit 16 (or other sensor) is present, the system may output the triggers but refrain from actually activating a new BP measurement.

In at least one embodiment, when the characteristics of the PPG signal are above or below defined normal thresholds or within or outside of defined ranges, the response triggering module 14 outputs the response trigger, such as the BP detection response, in order to indicate the potential need for new BP information at that particular time. As such, the response triggering module 14 may be in communication with the BP detection unit 16, which may activate the BP detection sensor 24, such as an NIBP cuff attached to patient anatomy. As shown in FIG. 1, the BP detection sensor 24 may be secured to an arm of the individual 20. Alternatively, the BP detection sensor 24 may be secured to various other physiological structures of the individual 20, such as a forehead, ear lobe, leg, etc. The BP detection unit 16 detects one or more BP values from the BP signal detected by the BP detection sensor 24. For example, based on the detected BP signal, the BP detection sensor 24 may determine systolic, diastolic, and mean arterial pressure (MAP) values of the individual 20. The BP may be detected based on any known techniques to non-invasively, or even invasively, detect BP. For example, the BP may be detected with the use of a standard BP cuff. Alternatively, the BP may be detected through an analysis of the PPG signal itself. In such an embodiment, the BP detection sensor 24 may include the PPG sensor 22 and/or another PPG sensor.

The BP variability index determination module 18 receives the number of response triggers from the response triggering module 14, and optionally the BP values from the BP detection unit 16. Additionally, the BP variability index determination module 18 may receive the times (for example, time stamped data) that the response triggers were output from the response triggering module 14. The output times of the response triggers may include the periods of time within a particular time window that the BP detection unit 16 is intended to be activated to detect the BP of the individual. As an example, the BP variability index determination module 18 may receive data signals related to the number and specific periods of time within a defined time window in which response triggers are output by the response triggering module 14. In at least one embodiment, the BP variability index determination module 18 may track the response triggers over a 1 hour time window. Alternatively, the time window may be more or less than 1 hour. For example, the time window may be 5 minutes, 10 minutes, 20 minutes, 30 minutes, or the like. The time window may be fixed, variable, or user-determined.

The BP variability index determination module 18 may determine a BP variability index based on a frequency of triggers—for example, the number of response triggers and the BP values associated with the response triggers within the time window. The BP values may be measured at or around the times the response triggers are output and stored in a memory, such as within the BP variability index determination module 18, and used to provide additional information related to the blood pressure stability and/or hemodynamic status of the individual 20. For example, the BP variability index may include a number of response triggers within a particular time window, as well as the BP values at or around the times the response triggers are output. Alternatively, the BP values may not be updated and/or may not be available.

As an example, a BP variability index may include A response triggers, B systolic pressure, C diastolic pressure, and D MAP, in which A is the number of response triggers within a time window, B is the systolic pressure value at or around the time each of the response triggers is output, C is the diastolic pressure value at or around the time each of the response triggers is output, and D is the MAP value at or around the time each of the response triggers is output. Alternatively, B, C, and D may be average or mean values. For example, if there are 3 response triggers within a particular time window, B, C, and D may be values that are average values calculated from BP values detected at or around the times the 3 response triggers were output.

The BP values may be detected within a time window (for example, a 1 hour time window) and then the variability of the BP values can be calculated. For example, the BP variability index determination module 18 may determine the BP variability of the BP values through statistical processes, such as comparison of maximum and minimum values, standard deviation of values, percentile ranges, and the like. The BP variability may be a measure of the frequency of the BP detection triggers.

The BP variability index may be used to determine a hemodynamic or health status of the individual 20. For example, if the number of response triggers within a time window (such as 1 minute) is 2 or less, the hemodynamic status of the individual 20 may be determined to be stable. If the number of response triggers within a time window is more than 2 but less than 4, the hemodynamic status of the individual 20 may be determined to be moderately stable or unstable. If the number of response triggers within a time window exceeds 4, the hemodynamic status of the individual 20 may be determined to be unstable or highly unstable. Alternatively, the time window may be more or less than 1 minute. For example, the time window may be 5 minutes, 10 minutes, an hour, etc. It should be noted that the BP variability index may be calculated even when no triggers are output. That is, the number of triggers within a time window may be zero, and that zero number may be used to calculate the BP variability index. In this case, the subject's blood pressure is presumed to be stable, and the BP variability index will be low. This information may still be useful, and thus the BP variability index can be reported continuously, even in the absence of triggers.

Additionally, the BP variability index may be based on a pattern of the response triggers. When the response triggers are evenly or regularly distributed or spaced within the time window, the triggers may indicate a regularly changing blood pressure. However, when triggers are clumped or clustered together in a group, with several triggers occurring in a short time period, the triggers may be more likely to be caused by interference in the signal, such as signal artifacts caused by patient movement or other noise sources. Accordingly, the pattern or distribution of triggers within the window may be used to calculate the BP variability index. When a high number of triggers are clustered together in a short duration, the triggers may be counted together as one trigger for purposes of calculating the BP variability index. As another example, when the BP variability index is calculated based on the high number of triggers, that index may be reduced when the triggers are clustered together. When the triggers are spaced apart from each other by a defined amount, the BP variability index may be reported based on the number or frequency of those triggers.

The pattern of triggers within a time window may also be used to determine confidence in the variability index. For example, if the response triggers are evenly and regularly spaced within the time window, the BP variability index may indicate a high degree of confidence with respect to the noted hemodynamic status (for example, stable, moderately stable/unstable, or highly unstable). If, however, some of the response trigger times are grouped together over a short period of time within the time window, the BP variability index may indicate a low degree of confidence with respect to the noted hemodynamic status, as the response triggers may have been output based on normal characteristic changes of the PPG signal due to patient movement, coughing, or the like. For example, if the BP variability index determination module 18 detects 5 response triggers within a 1 hour time window, but 3 of the 5 response triggers occurred within 1 minute, the BP variability index determination module 18 may output a BP variability index indicating a low degree of confidence in the variability value.

As noted, the BP variability index may also include the BP values detected by the BP detection unit 16 at or around the times the response triggers are output. The BP values may be coupled to the number and/or pattern of response triggers to provide additional details regarding the hemodynamic or health status of the individual 20. For example, if the BP variability index determination module 18 detects a moderate number of response triggers within a particular time window (such as 3 triggers within an hour), and the BP values at or around the output times of the response triggers exceed normal BP thresholds, then the BP variability index determination module 18 may output a BP variability index that indicates a moderate stability at heightened BP values. As another example, if the BP variability index determination module 18 detects a large number of response triggers within a particular time window (such as 5 triggers within an hour), but the BP values associated with the 5 response triggers are within an acceptable percentage of safe BP thresholds (for example, within 0-5% of safe BP thresholds), then the BP variability index determination module 18 may output a BP variability index that indicates instability at relatively safe BP values. In short, the BP variability index may be based on a number and/or output times of the response triggers and the BP values associated with the response triggers. Additional information may also be included with the BP variability index. For example, the BP variability index may be displayed with information regarding qualifications, caveats, and/or instructions to confirm through continued monitoring, testing, and/or the like.

The response triggering module 14 may output response triggers and/or calibrations based on analysis of the PPG signal received from the PPG sub-system 12 as described, for example, in United States Patent Application Publication No. 2012/0143067, entitled “Systems and Methods for Determining When to Measure a Physiological Parameter,” United States Patent Application Publication No. 2012/0143012, entitled “Systems and Methods for Physiological Event Marking,” United States Patent Application Publication No. 2010/0081892, entitled “Systems and Methods for Combined Pulse Oximetry and Blood Pressure Measurement,” all of which are hereby incorporated by reference in their entireties. Optionally, the response triggers may be based on a fluid responsiveness parameter or predictor (FRP), such as described below.

The response triggering module 14 and the BP variability index determination module 18 may include one or more control units, circuits, or the like, such as processing devices that may include one or more microprocessors, microcontrollers, integrated circuits, memory, such as read-only and/or random access memory, and the like. As an example, each of the modules 14 and 18 may include or be formed as an integrated chip. Each of the modules 14 and 18 may be separate and distinct circuits or processors within the system 10, for example. Optionally, the modules 14 and 18 may be integrated into a single circuit or processor.

The modules 14 and 18 may be contained within a workstation that may be or otherwise include one or more computing devices, such as standard computer hardware (for example, processors, circuitry, memory, and the like). The PPG sub-system 12 may be operatively connected to the workstation, such as through a cable or wireless connection. While the PPG sub-system 12 and the modules 14 and 18 are shown as separate components, the PPG sub-system 12 and the modules 14 and 18 may be integrally part of a single unit, workstation, or the like. As an example, the modules 14 and 18 may be integrally part of the PPG sub-system 12. Optionally, the modules 14 and 18 and the PPG sub-system 12 may all be contained within a single housing or workstation that operatively connects to the PPG sensor 22.

Additionally, one or both of the PPG sub-system 12 and the modules 14 and 18 may be housed within a smart cable, adapter, or the like, that is part of a cable assembly having one or more sensors at one end, and a connector configured to connect to a monitor at an opposite end. In this manner, the PPG sub-system 12 and/or the modules 14 and 18 may be configured to connect to a device configured to display the BP variability index to an individual. For example, the PPG sub-system 12 and/or the modules 14 and 18 may be part of an assembly that connects to a device, such as a cellular or smart phone, tablet, other handheld device, laptop computer, monitor, or the like that may be configured to receive data from the assembly and show the data on a display of the device. In an embodiment, the device may be configured to download software in the form of applications configured to operate in conjunction with the assembly.

The system 10 may include any suitable computer-readable media used for data storage. For example, the modules 14 and 18 may include computer-readable media. The computer-readable media are configured to store information that may be interpreted by the modules 14 and 18. The information may be data or may take the form of computer-executable instructions, such as software applications, that cause a microprocessor or other such control unit within the modules 14 and 18 to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

FIG. 2 illustrates a simplified block diagram of a system 30 for determining a hemodynamic status of an individual 31, according to an embodiment of the present disclosure. The system 30 may include a PPG sub-system 32 in communication with a response triggering module 34 that is in communication with a BP variability index determination module 36. The system 30 is similar to the system 10, except that the system 30 may not include a BP detection unit (such as the BP detection unit 16 show in FIG. 1).

In the embodiment shown in FIG. 2, the number of the response triggers themselves within a time window may be used to determine a hemodynamic stability of the individual. For example, the BP variability index determination module 36 may determine a BP variability index based simply on the number of response triggers output from the response triggering module 34 within a time window. Based on the analysis of the PPG signal, the response triggering module 34 may output the response triggers even though the system 30 does not include a BP detection unit. The response triggers are output at times in which a BP reading would be suggested, due to the changing characteristics of the PPG signal output by the PPG sub-system 32. However, while the response triggers are output by the response triggering module 34, the BP of the individual need not actually be measured. Instead, the response triggers themselves, which may be related to an instruction to initiate detection of BP, may be counted over a time window to determine a BP variability index. Thus, a BP variability index may be determined without the BP of the patient actually being measured.

For example, if the response triggering module 34 outputs 2 or less response triggers within a particular time window, such as 1 hour, the BP variability index determination module 36 may output a BP variability index indicating 2 response triggers and/or an indication of hemodynamic stability. In such an example, 2 response triggers within 1 hour may be associated with a stable hemodynamic state. If, however, the response triggering module 34 outputs 3 or 4 response triggers within the time window, the BP variability index determination module 36 may output a BP variability index indicating 3 or 4 response triggers and/or an indication of moderate hemodynamic instability. In this example, 3 or 4 response triggers within the time window may be associated with a moderate hemodynamic instability. If the response triggering module 34 outputs 5 or more response triggers within the time window, the BP variability index determination module 36 may output a BP variability index indicating 5 or more response triggers and/or an indication of a high degree of hemodynamic instability. In this example, 5 or more response triggers within the time window may be associated with a high degree of hemodynamic instability. It is to be understood that the thresholds for stability, moderate instability, and a high degree of instability noted above are merely examples. The thresholds may be greater or less than described. Additionally, the time window may be greater or less than 1 hour.

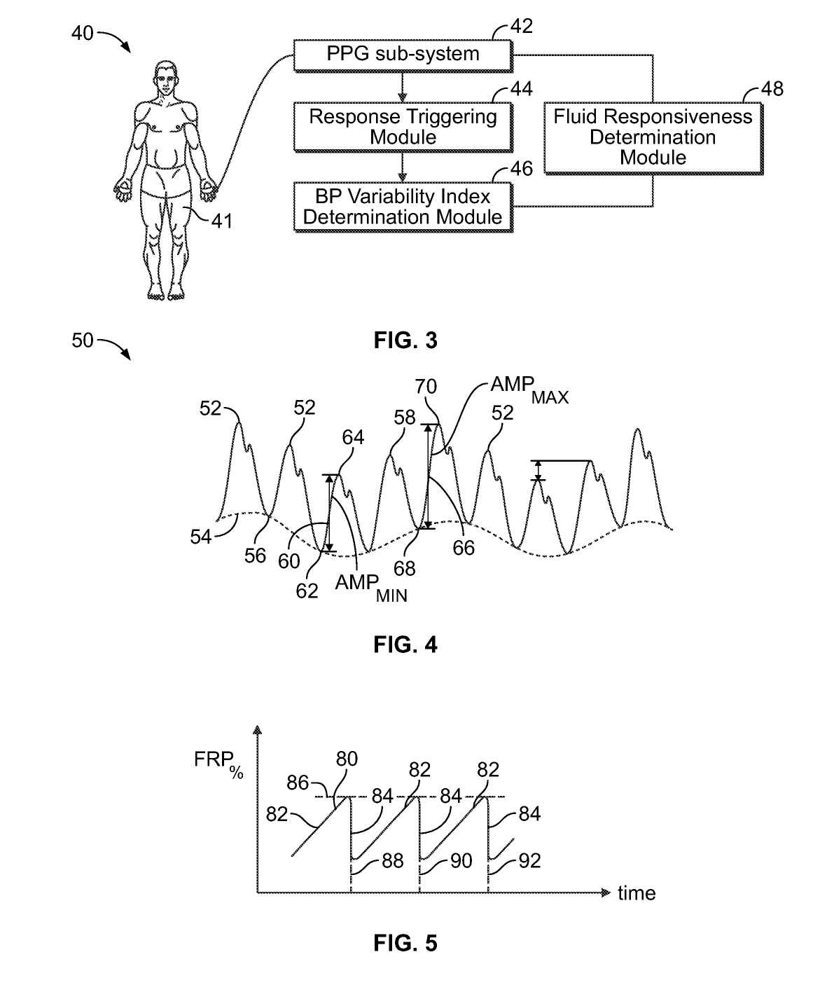

FIG. 3 illustrates a simplified block diagram of a system 40 for determining a hemodynamic status of an individual 41, according to an embodiment of the present disclosure. The system 40 may include a PPG sub-system 42 in communication with a response triggering module 44 that is in communication with a BP variability index determination module 46. While not shown, the system 40 may also include a BP detection unit.

The system 40 is similar to the systems 10 and 30 described above, except that the system 40 includes a fluid responsiveness determination module 48 that may be in communication with the PPG sub-system 42 and/or the BP variability index determination module 46. In at least one embodiment, a frequency of output response triggers within a particular time window may be used to determine whether to administer fluid to an individual and may be used to diagnose complications such as hypovolemia and/or persistent hemorrhaging.

In at least one embodiment, the fluid responsiveness determination module 48 analyzes the PPG signal output from the PPG sub-system 42 to determine a respiratory variation of the PPG signal, which correlates with fluid responsiveness. Respiratory variation in the arterial blood pressure waveform is known to be a good predictor of a patient's response to fluid loading, or fluid responsiveness. Fluid responsiveness represents a prediction of whether such fluid loading will improve blood flow within the patient. Fluid responsiveness refers to the response of stroke volume or cardiac output to fluid administration. A patient is said to be fluid responsive if fluid loading does accomplish improved blood flow, such as by an improvement in cardiac output or stroke volume index by about 10%, 15%, or another percentage, as appropriate. Fluid is delivered with the expectation that it will increase the patient's cardiac preload, stroke volume, and cardiac output, resulting in improved oxygen delivery to the organs and tissue. Fluid delivery may also be referred to as volume expansion, fluid therapy, fluid challenge, or fluid loading. Monitoring fluid responsiveness allows a physician to determine whether additional fluid should be provided to an individual, such as through an intravenous fluid injection.

FIG. 4 illustrates a representation of a PPG signal 50, according to an embodiment of the present disclosure. The PPG signal 50 includes cardiac pulses 52 superimposed on a baseline 54. The baseline 54 may be defined as extending from and between pulse minimums 56, such as troughs or valleys, of the PPG signal 50. Optionally, depending on how the PPG signal 50 is filtered, a baseline may be defined as extending from and between pulse maximums 58 of the PPG signal 50. Also, alternatively, a baseline may be defined as extending from and between locations of the PPG signal 50 that are between the pulse minimums 56 and the pulse maximums 58.

Referring to FIGS. 3 and 4, the fluid responsiveness determination module 48 may analyze the PPG signal 50 to determine a respiratory variation of the PPG signal 50, which may be used as the FRP. For example, the fluid responsiveness determination module 48 may analyze the PPG signal 50 to determine an FRP, such as ΔPOP.

In at least one embodiment, ΔPOP is calculated as follows. The fluid responsiveness determination module 48 may measure an amplitude AMP_{MIN} of a minimum cardiac pulse 60 from a pulse minimum 62 to a pulse maximum 64. Similarly, an amplitude AMP_{MAX} of a maximum cardiac pulse 66 may be measured from a pulse minimum 68 to a pulse maximum 70. The respiratory variation or modulation causes the upstroke amplitude values (AMP) to vary cyclically over each breath.

AMP_{MAX} and AMP_{MIN} may be identified with respect to the PPG signal 50 in a particular time window. AMP_{MAX} may be the greatest amplitude of a pulse upstroke within the time window, while AMP_{MIN} may be the smallest amplitude of a pulse upstroke within the time window. The fluid responsiveness determination module 48 may identify AMP_{MAX} and AMP_{MIN} and input both into the following:

ΔPOP=(AMP_{MAX}−AMP_{MIN})/AMP_{AVG} Equation (1)

where

AMP_{AVG}=(AMP_{MAX}+AMP_{MIN})/2 Equation (2)

ΔPOP may define the respiratory variation in the amplitude of the PPG signal 50. ΔPOP is a unit-less value, such a number or percentage. Based on the FRP, such as ΔPOP, a determination of whether or not to administer fluid to an individual may be made. For example, if ΔPOP is above a particular threshold, such as 15%, a clinician may determine that the individual would benefit from the administration of fluid. The 15% threshold is merely an example, and it is to be understood that the threshold may be greater or less than 15%. Moreover, a particular threshold may be used to determine that an individual would benefit from fluid administration, or that fluid administration should cease.

ΔPOP represents just one example of a FRP. Various other FRPs may be used. In other embodiments, the FRP metric is a measure of the respiratory variation of the PPG, such as a measure of the baseline modulation of the PPG, or other suitable metrics assessing the respiratory modulation of the PPG. For example, an FRP may be based on the amplitudes or areas of acceptable cardiac pulses 52 within a particular time frame or window. The minimum amplitude of the cardiac pulses 52 may be subtracted from the maximum amplitude then divided by an average or mean value. Alternatively, an FRP may be derived from a frequency of cardiac pulses 52 within a time frame or window. For example, a modulation or variation in frequency among two or more cardiac pulses may be used to derive an FRP. In general, the FRP may be based on one or more respiratory variations exhibited by the PPG signal 50. Further, a FRP may be determined through the use of wavelet transforms, such as described in United States Patent Application Publication No. 2010/0324827, entitled “Fluid Responsiveness Measure,” which is hereby incorporated by reference in its entirety.

Also, alternatively, the fluid responsiveness determination module 48 may determine a FRP based on pulse pressure variation, pulse wave velocity, reflected pulse intensity, respiratory sinus arrhythmia (RSA), and/or the like. Other signals may be used to derive an FRP including a blood pressure signal, stroke volumes, aortic and other blood flow velocities, ETCO₂ (end tidal CO₂) signals, photoacoustic signals, and the like.

Moreover, an FRP, such as ΔPOP, may be used in connection with a response trigger. For example, if the FRP is above or below a certain threshold or inside or outside of a range, the response triggering module may output a response trigger that is configured to instruct initiation of blood pressure detection.

FIG. 5 illustrates a chart of a FRP 80 over time, according to an embodiment of the present disclosure. As shown, the FRP 80 may exhibit steady increases 82 punctuated by sharp decreases 84. Each of the sharp decreases 84 may be the result of fluids being administered to an individual, which may thereby decrease the FRP 80. The saw-tooth pattern of the FRP 80 shown in FIG. 5, characterized by the steady increases 82 and sharp decreases 84, may be indicative of a hypovolemic state. The FRP 80 increases as hypovolemia becomes more acute. For example, as an individual loses fluid volume, the FRP 80 increases. As shown in FIG. 5, as the individual loses fluid volume, the FRP 80 steadily increases 82 to a threshold 86 in which it is determined that fluid should be administered to the individual. When fluid is administered to the individual at times 88, 90 and 92, the FRP 80 sharply decreases 84. However, because the individual continues to lose fluid volume, the FRP again steadily increases 82 almost immediately after the fluid is administered, thereby indicating that the individual is in a hypovolemic state.

Referring to FIGS. 3 and 5, when the FRP 80 meets or exceeds the threshold 86, the response triggering module 44 may output a response trigger, such as a BP or FRP detection response. In this manner, the response trigger may be based on the FRP 80. The threshold 86 may be greater or less than shown. As the FRP 80 steadily increases 82, the FRP eventually meets the threshold 86, which generates the response trigger. The threshold 86 may be greater or less than a threshold used in a determination regarding the administration of fluids to an individual.

The fluid responsiveness determination module 48 may determine that an individual is suffering from hypovolemia or blood loss based on a repeating pattern of the FRP over time, such as the saw-tooth waveform shown in FIG. 5. For example, if a recurring pattern of the FRP 80 exhibiting a steadily increase 82 followed by a sharp decrease 84 upon fluid administration (such as 2, 3, 4, or more repeating patterns) appears, then the fluid responsiveness determination module 48 and/or the BP variability index determination module 46 may determine and indicate a hypovolemic state.

The BP variability index determination module 46 may determine a BP variability index, such as with respect to any of the embodiments described above, and couple that determination with a status of the FRP over time. For example, the BP variability index may be analyzed in conjunction with a repeating FRP pattern, as shown in FIG. 5, to determine that an individual is suffering from hypovolemia. For example, if a BP variability index is determined to be stable, and there is little to no increase in FRP 80, then the individual may be determined to be hemodynamically stable. If, however, the BP variability index is determined to be moderately unstable, and the FRP 80 exhibits a mildly or moderately modulating pattern over the same time period (for example, between 2 and 4 incidences of a steady increase 82 followed by a sharp decrease 84), the individual may be diagnosed as mildly hypovolemic. If the BP variability index is determined to be highly unstable, and the FRP exhibits a clear or severe modulating pattern (for example, 5 or more steady increases 82 followed by sharp decreases 84) over the same time period, the individual may be diagnosed as highly hypovolemic. As such, the BP variability index coupled with a FRP status or pattern over time may be used to determine a hemodynamic status of an individual.

Additionally, the BP variability index and the FRP may be used as redundancy or accuracy checks. For example, if the BP variability index indicates hemodynamic stability, but the FRP 80 exhibits a saw-tooth repeating pattern as shown in FIG. 5, which may indicate hypovolemia, the system 40 may emit a visual and/or audio alert regarding inconsistent results.

Referring again to FIG. 3, the fluid responsiveness determination module 48 may include one or more control units, circuits, or the like, such as processing devices that may include one or more microprocessors, microcontrollers, integrated circuits, memory, such as read-only and/or random access memory, and the like. The module 48 may be contained within a workstation that may be or otherwise include one or more computing devices, such as standard computer hardware (for example, processors, circuitry, memory, and the like). The PPG sub-system 42 may be operatively connected to the workstation, such as through a cable or wireless connection. While the PPG sub-system 42 and the module 48 are shown as separate components, the PPG sub-system 42 and the module 48 may be integrally part of a single unit, workstation, or the like. As an example, the module 48 may be integrally part of the PPG sub-system 42. Additionally, one or both of the PPG sub-system 42 and the module 48 may be housed within a smart cable, adapter, or the like, that is part of a cable assembly having one or more sensors at one end, and a connector configured to connect to a monitor at an opposite end.

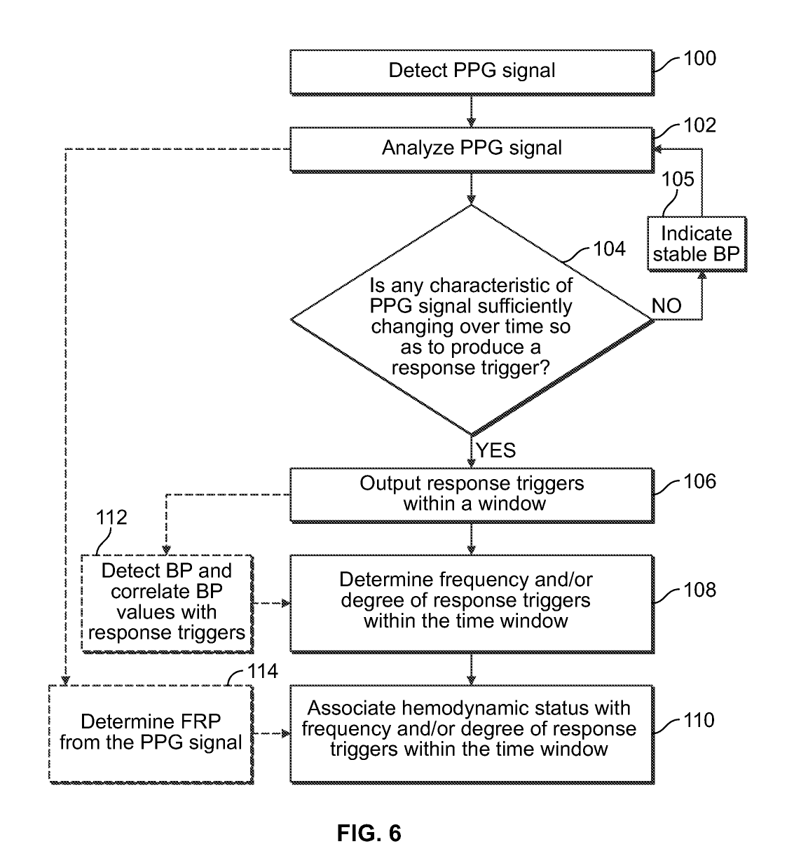

FIG. 6 illustrates a flow chart of a method for determining a hemodynamic status of an individual, according to an embodiment of the present disclosure. The method begins at 100, in which a PPG signal is detected, such as through a PPG sub-system. At 102, the PPG signal is analyzed, such as with a response triggering module. At 104, it is determined if any characteristic of the PPG signal is sufficiently changing over time so as to produce a response trigger. If not, the process continues to 105, in which a stable BP is indicated (such as on a display or monitor), and then the process returns to 102.

If, however, at least one characteristic of the PPG signal (such as an amplitude or frequency of an AC or DC component of the PPG signal) is changing in relation to a threshold, then the method continues to 106, in which the response triggering module outputs one or more response triggers within a time window. The method may then proceed to 108, in which the frequency and/or degree of response triggers within the time window is determined so as to form a BP variability index. As noted, the BP variability index may be calculated even if no response triggers are output. That is, if zero response triggers are output, then the BP is stable, and the BP variability index is low. At 110, hemodynamic status is associated with the frequency and/or degree of response triggers within the time window. For example, a BP variability index value of 5 response triggers within an hour may be associated with a hemodynamically unstable condition or state.

Alternatively, concurrent with or after the output response triggers are output at 106, a BP of an individual may be detected and BP values may be correlated with the output times of particular response triggers at 112. The process may then proceed to 108, in which the BP values may be used to generate the BP variability index in conjunction with the frequency and/or degree of the response triggers.

Also, alternatively, subsequent to 102, a FRP may be determined based on an analysis of the PPG signal at 114. The FRP and the BP variability index, which may be based on the frequency and/or degree of the response triggers, may then, in combination, form a BP variability index and associated with a particular hemodynamic status. For example, an initial BP variability index may be supplemented, clarified, augmented, or the like by the FRP to form a final BP variability index that may be associated with a particular hemodynamic status. The FRP and the initial BP variability index may be viewed together to determine or indicate a final BP variability index. Alternatively, the FRP and the initial BP variability index may be combined together into a single final BP variability index. For example, a numerical value of the FRP may be averaged with, added to, subtracted from, multiplied with, or divided by a numerical value of the initial BP variability index to yield the final BP variability index. Further, the FRP may be used as a response trigger, as described above.

United States Patent Application Publication No. 2012/0143067, entitled “Systems and Methods for Determining When to Measure a Physiological Parameter,” which is hereby incorporated by reference in its entirety, discloses systems and methods for determining when to update a blood pressure measurement. United States Patent Application Publication No. 2012/0143012, entitled “Systems and Methods for Physiological Even Marking,” which is hereby incorporated by reference in its entirety, discloses patient monitoring systems that may store an event marker, trigger a response, update a metric value, or the like. United States Patent Application Publication No. 2010/0081892, entitled “Systems and Methods for Combined Pulse Oximetry and Blood Pressure Measurement,” which is hereby incorporated by reference in its entirety, relates to a combined sensor that includes a pulse oximetry sensor component and a continuous non-invasive blood pressure sensor component.

Embodiments of the present disclosure provide systems and methods for generating a BP variability index that provides information regarding patent illness severity and/or hemodynamic status. Certain embodiments may employ a NIBP cuff may that automatically activates (or triggers) based on output response triggers that are generated when changes in hemodynamic status are detected. Alternatively, embodiments of the present disclosure may not include a NIBP cuff, or any other separate and distinct device that is specifically configured to detect BP.

With reference to any of the embodiments described above, statistical metrics may be normalized with respect to a baseline (for example, the mean, median, or mode of the measurements) to provide the BP variability index. For example, particular thresholds may be determined based on empirical or clinical data that relate to hemodynamically stable, moderately unstable, and highly unstable states. Embodiments of the present disclosure may include monitors and/or speakers that generate visual and/or audio alarms for various levels of hemodynamic instability.

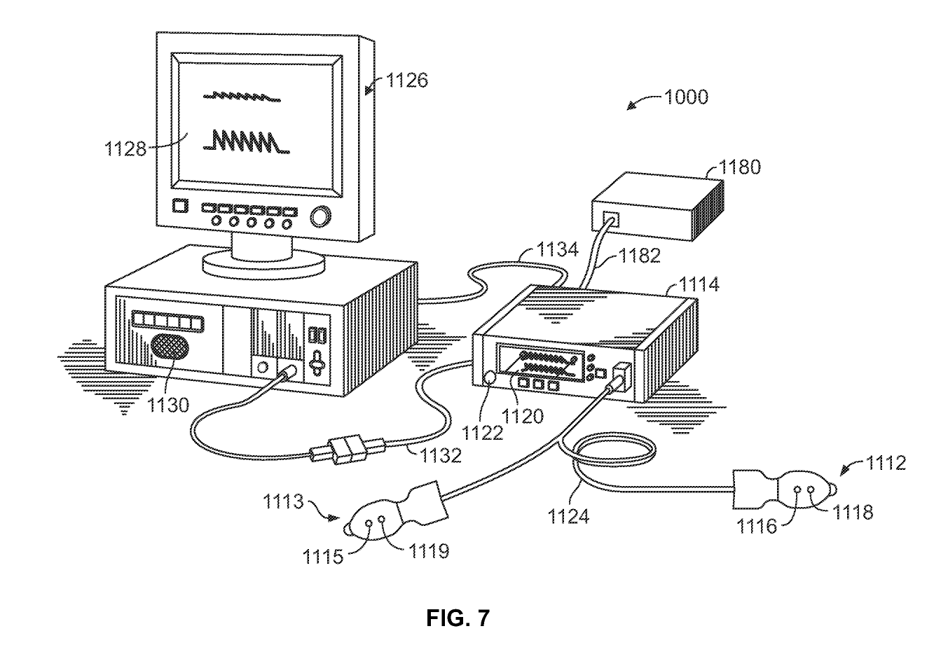

FIG. 7 illustrates a perspective view of a monitoring system 1000, according to an embodiment of the present disclosure. The system 1000 may be an example of, or include, any of the PPG sub-systems described above. The system 1000 may include a sensor unit 1112 and a monitor 1114. In at least one embodiment, the sensor unit 1112 may be part of a continuous, non-invasive blood pressure (CNIBP) monitoring system and/or an oximeter. The sensor unit 1112 may include an emitter 1116 for emitting light at one or more wavelengths into an individual's tissue. A detector 1118 may also be provided in the sensor 1112 for detecting the light originally from emitter 1116 that emanates from patient tissue after passing through the tissue. Any suitable physical configuration of the emitter 1116 and the detector 1118 may be used. In at least one embodiment, the sensor unit 1112 may include multiple emitters and/or detectors, which may be spaced apart. The system 1000 may also include one or more additional sensor units, such as sensor unit 1113, which may take the form of any of the embodiments described herein with reference to the sensor unit 1112. For example, the sensor unit 1113 may include an emitter 1115 and a detector 1119. The sensor unit 1113 may be the same type of sensor unit as the sensor unit 1112, or the sensor unit 1113 may be of a different sensor unit type than the sensor unit 1112. The sensor units 1112 and 1113 may be capable of being positioned at two different locations on a subject's body. For example, the sensor unit 1112 may be positioned on an individual's forehead, while the sensor unit 1113 may be positioned at an individual's fingertip.

According to at least one embodiment, the emitter 1116 and the detector 1118 may be on opposite sides of a digit such as a finger or toe, in which case the light that is emanating from the tissue has passed completely through the digit. In an embodiment, the emitter 1116 and the detector 1118 may be arranged so that light from the emitter 1116 penetrates the tissue and is reflected by the tissue into the detector 1118, such as in a sensor designed to obtain pulse oximetry data from an individual's forehead.

In at least one embodiment, the sensor unit 1112 may be connected to and draw its power from the monitor 1114, as shown. In at least one other embodiment, the sensor unit 1112 may be wirelessly connected to the monitor 1114 and include its own battery or similar power supply (not shown). The monitor 1114 may be configured to calculate physiological characteristics or parameters (e.g., pulse rate, blood pressure, blood oxygen saturation) based at least in part on data relating to light emission and detection received from one or more sensor units such as the sensor units 1112 and 1113. Alternatively, the calculations may be performed on the sensor units or an intermediate device and the result of the calculations may be passed to the monitor 1114. Further, the monitor 1114 may include a display 1120 configured to display the physiological parameters or other information about the system 1000. In the embodiment shown, the monitor 1114 may also include a speaker 1122 to provide an audible sound that may be used in various other embodiments, such as for example, sounding an audible alarm in the event that an individual's physiological parameters are not within a predefined normal range. In an embodiment, the monitor 1114 includes a blood pressure monitor. In alternative embodiments, the system 1000 includes a stand-alone blood pressure monitor in communication with the monitor 1114 via a cable or a wireless network link.

In an embodiment, the sensor unit 1112 may be communicatively coupled to the monitor 1114 via a cable 1124. However, in other embodiments, a wireless transmission device (not shown) or the like may be used instead of or in addition to cable 1124.

The system 1000 may include a multi-parameter patient monitor 1126. The monitor 1126 may include a cathode ray tube display, a flat panel display (as shown) such as a liquid crystal display (LCD) or a plasma display, or may include any other type of monitor now known or later developed. The multi-parameter patient monitor 1126 may be configured to calculate physiological parameters and to provide a display 1128 for information from the monitor 1114 and from other medical monitoring devices or systems (not shown). For example, the multi-parameter patient monitor 1126 may be configured to display an estimate of an individual's blood oxygen saturation generated by the monitor 1114 (referred to as a “SpO₂” measurement), pulse rate information from the monitor 114 and blood pressure from the monitor 1114 on the display 1128. The multi-parameter patient monitor 1126 may also include a speaker 1130.

The monitor 1114 may be communicatively coupled to the multi-parameter patient monitor 1126 via a cable 1132 or 1134 that is coupled to a sensor input port or a digital communications port, respectively and/or may communicate wirelessly (not shown). In addition, the monitor 1114 and/or the multi-parameter patient monitor 1126 may be coupled to a network to enable the sharing of information with servers or other workstations (not shown). The monitor 1114 may be powered by a battery (not shown) or by a conventional power source such as a wall outlet.

A calibration device 1180, which may be powered by the monitor 1114 via a cable 1182, a battery, or by a conventional power source such as a wall outlet, may include any suitable signal calibration device. The calibration device 1180 may be communicatively coupled to the monitor 1114 via cable 1182, and/or may communicate wirelessly (not shown). In other embodiments, the calibration device 1180 is completely integrated within the monitor 1114. For example, the calibration device 1180 may take the form of any invasive or non-invasive blood pressure monitoring or measuring system used to generate reference blood pressure measurements for use in calibrating a CNIBP monitoring technique. Such calibration devices may include, for example, an aneroid or mercury sphygmomanometer and occluding cuff, a pressure sensor inserted directly into a suitable artery of an individual, an oscillometric device or any other device or mechanism used to sense, measure, determine, or derive a reference blood pressure measurement. In some embodiments, the calibration device 180 may include a manual input device (not shown) used by an operator to manually input reference signal measurements obtained from some other source (e.g., an external invasive or non-invasive physiological measurement system).

The calibration device 1180 may also access reference signal measurements stored in memory (e.g., RAM, ROM, or a storage device). For example, in some embodiments, the calibration device 1180 may access reference blood pressure measurements from a relational database stored within the calibration device 1180, the monitor 1114, or the multi-parameter patient monitor 1126. The reference blood pressure measurements generated or accessed by the calibration device 1180 may be updated in real-time, resulting in a continuous source of reference blood pressure measurements for use in continuous or periodic calibration. Alternatively, reference blood pressure measurements generated or accessed by the calibration device 1180 may be updated periodically, and calibration may be performed on the same periodic cycle or a different periodic cycle. Reference blood pressure measurements may be generated when recalibration is triggered.

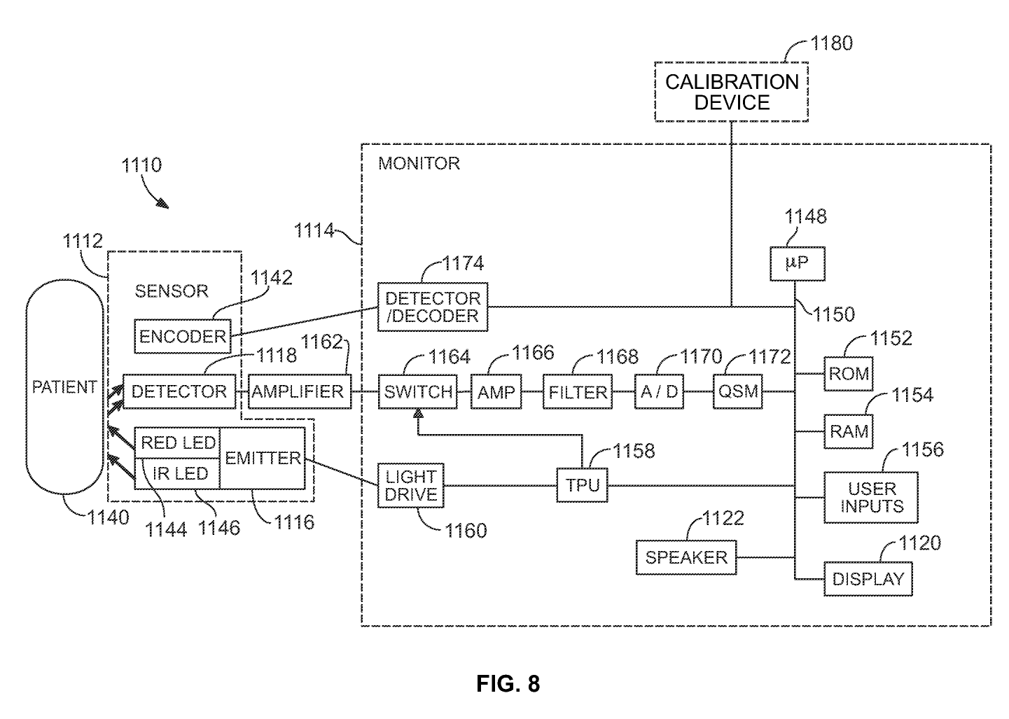

FIG. 8 illustrates a block diagram of a monitoring system 1110, such as the monitoring system 1000 of FIG. 7, according to an embodiment of the present disclosure. The system 1110 may be coupled to an individual 1140. Because the sensor units 1112 and 1113 may include similar components and functionality, only the sensor unit 1112 will be discussed in detail for ease of illustration. It will be understood that any of the concepts, components, and operation discussed in connection with the sensor unit 1112 may be applied to the sensor unit 1113 as well (e.g., the emitter 1116 and the detector 1118 of the sensor unit 1112 may be similar to the emitter 1115 and the detector 1119 of the sensor unit 1113). It will be noted that the system 1110 may include one or more additional sensor units or probes, which may take the form of any of the embodiments described herein with reference to the sensor units 1112 and 1113. These additional sensor units included in the system 1110 may take the same form as the sensor unit 1112, or may take a different form. In an embodiment, multiple sensors (distributed in one or more sensor units) may be located at multiple different body sites on an individual.

The sensor unit 1112 may include the emitter 1116, the detector 1118, and an encoder 1142. In the embodiment shown, the emitter 1116 may be configured to emit at least two wavelengths of light (e.g., Red and IR) into an individual's tissue 1140. Hence, the emitter 1116 may include a Red light emitting light source such as Red light emitting diode (LED) 1144 and an IR light emitting light source such as IR LED 1146 for emitting light into the individual's tissue 1140 at the wavelengths used to calculate the individual's physiological parameters. In one embodiment, the Red wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a Red light while a second emits only an IR light. In another example, the wavelengths of light used are selected based on the specific location of the sensor.

It will be understood that, as used herein, the term “light” may refer to energy produced by radiation sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. The detector 1118 may be chosen to be specifically sensitive to the chosen targeted energy spectrum of the emitter 116.

In at least one embodiment, the detector 1118 may be configured to detect the intensity of light at the Red and IR wavelengths. Alternatively, each sensor in the array may be configured to detect an intensity of a single wavelength. In operation, light may enter the detector 1118 after passing through the individual's tissue 1140. The detector 1118 may convert the intensity of the received light into an electrical signal. The light intensity is directly related to the absorbance and/or reflectance of light in the tissue 1140. That is, when more light at a certain wavelength is absorbed or reflected, less light of that wavelength is received from the tissue by the detector 1118. After converting the received light to an electrical signal, the detector 1118 may send the signal to the monitor 1114, where physiological parameters may be calculated based on the absorption of the Red and IR wavelengths in the individual's tissue 1140.

In at least one embodiment, the encoder 1142 may contain information about the sensor unit 1112, such as what type of sensor it is (e.g., whether the sensor is intended for placement on a forehead or digit) and the wavelengths of light emitted by the emitter 1116. This information may be used by the monitor 1114 to select appropriate algorithms, lookup tables and/or calibration coefficients stored in the monitor 1114 for calculating the individual's physiological parameters.

The encoder 1142 may contain information specific to the individual 1140, such as, for example, the individual's age, weight, and diagnosis. This information about an individual's characteristics may allow the monitor 1114 to determine, for example, patient-specific threshold ranges in which the individual's physiological parameter measurements should fall and to enable or disable additional physiological parameter algorithms. This information may also be used to select and provide coefficients for equations from which, for example, blood pressure and other measurements may be determined based at least in part on the signal or signals received at the sensor unit 1112. For example, some pulse oximetry sensors rely on equations to relate an area under a pulse of a PPG signal to determine blood pressure. These equations may contain coefficients that depend upon an individual's physiological characteristics as stored in the encoder 1142. The encoder 1142 may, for instance, be a coded resistor which stores values corresponding to the type of sensor unit 1112 or the type of each sensor in the sensor array, the wavelengths of light emitted by the emitter 1116 on each sensor of the sensor array, and/or the individual's characteristics. In another embodiment, the encoder 1142 may include a memory on which one or more of the following information may be stored for communication to the monitor 1114: the type of the sensor unit 1112; the wavelengths of light emitted by the emitter 1116; the particular wavelength each sensor in the sensor array is monitoring; a signal threshold for each sensor in the sensor array; any other suitable information; or any combination thereof.

In at least one embodiment, signals from the detector 1118 and the encoder 1142 may be transmitted to the monitor 1114. In the embodiment shown, the monitor 1114 may include a general-purpose microprocessor 1148 connected to an internal bus 1150. The microprocessor 1148 may include, for example, any of the response triggering modules, BP variability index determination modules, or fluid responsiveness determination modules described above. The microprocessor 1148 may be adapted to execute software, which may include an operating system and one or more applications, as part of performing the functions described herein. Also connected to the bus 1150 may be a read-only memory (ROM) 1152, a random access memory (RAM) 1154, user inputs 1156, display 1120, and speaker 1122.

RAM 1154 and ROM 1152 are illustrated by way of example, and not limitation. Any suitable computer-readable media may be used in the system for data storage. Computer-readable media are capable of storing information that can be interpreted by the microprocessor 1148. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 1158 may provide timing control signals to light drive circuitry 1160, which may control when the emitter 1116 is illuminated and multiplexed timing for Red LED 1144 and IR LED 1146. TPU 1158 may also control the gating-in of signals from the detector 1118 through amplifier 1162 and switching circuit 1164. These signals are sampled at the proper time, depending upon which light source is illuminated. The received signal from detector 1118 may be passed through amplifier 1166, low pass filter 1168, and analog-to-digital converter 1170. The digital data may then be stored in a queued serial module (QSM) 1172 (or buffer) for later downloading to RAM 1154 as QSM 1172 fills up. In at least one embodiment, there may be multiple separate parallel paths having components equivalent to amplifier 1166, filter 1168, and/or A/D converter 1170 for multiple light wavelengths or spectra received.

In at least one embodiment, the microprocessor 1148 may determine the individual's physiological characteristics or parameters, such as SpO₂, pulse rate, and/or blood pressure, using various algorithms and/or look-up tables based on the value of the received signals and/or data corresponding to the light received by the detector 1118. Signals corresponding to information about the individual 1140, and particularly about the intensity of light emanating from an individual's tissue over time, may be transmitted from the encoder 1142 to decoder 1174. These signals may include, for example, encoded information relating to patient characteristics. The decoder 1174 may translate these signals to enable the microprocessor 1148 to determine the thresholds based at least in part on algorithms or look-up tables stored in ROM 1152. User inputs 1156 may be used to enter information about the individual, such as age, weight, height, diagnosis, medications, treatments, and so forth. In at least one embodiment, the display 1120 may exhibit a list of values which may generally apply to the individual, such as, for example, age ranges or medication families, which the user may select using user inputs 1156.

Pulse oximeters, in addition to providing other information, can be utilized for continuous non-invasive blood pressure monitoring. For example, PPG and other pulse signals obtained from multiple probes can be processed to calculate the blood pressure of an individual. In particular, blood pressure measurements may be derived based on a comparison of time differences between certain components of the pulse signals detected at each of the respective probes. As described in U.S. Patent Application Publication No. 2009/0326386, entitled “Systems and Methods For Non-Invasive Blood Pressure Monitoring,” the entirety of which is incorporated herein by reference, blood pressure can also be derived by processing time delays detected within a single PPG or pulse signal obtained from a single pulse oximeter probe. In addition, as described in U.S. Pat. No. 8,398,556, entitled “Systems and Methods For Non-Invasive Continuous Blood Pressure Determination,” the entirety of which is incorporated herein by reference, blood pressure may also be obtained by calculating the area under certain portions of a pulse signal. Also, as described in U.S. Patent Application Publication No. 2010/0081945, entitled “Systems and Methods for Maintaining Blood Pressure Monitor Calibration,” the entirety of which is incorporated herein by reference, a blood pressure monitoring device may be recalibrated in response to arterial compliance changes.

Blood pressure detection may be activated based on various response triggers that relate to changes in at least one characteristic of the PPG signals. For example, when an FRP that is based on a PPG signal exceeds a particular threshold, the system 1110 may be triggered to detect a blood pressure of the individual. Notably, the blood pressure of the individual may be detected in various non-invasive and invasive systems and methods.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

Referring to FIGS. 1-8, the various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer” or “module.”

The computer or processor executes a set of instructions that are stored in one or more storage elements, in order to process data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

The block diagrams of embodiments herein illustrate one or more modules. It is to be understood that the modules represent circuit modules that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hardwired to perform the functions described herein. Optionally, the hardware may include electronic circuits that include and/or are connected to one or more logic-based devices, such as microprocessors, processors, controllers, or the like. Optionally, the modules may represent processing circuitry such as one or more field programmable gate array (FPGA), application specific integrated circuit (ASIC), or microprocessor. The circuit modules in various embodiments may be configured to execute one or more algorithms to perform functions described herein. The one or more algorithms may include aspects of embodiments disclosed herein, whether or not expressly identified in a flowchart or a method.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, lower, mid, lateral, horizontal, vertical, front, and the like may be used to describe embodiments of the present disclosure, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” may be used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112(f) unless and until such claim limitations expressly use the phrase “means for” followed by a statement of function void of further structure.

What is claimed is:

1. A system for determining a hemodynamic status of an individual, the system comprising:
a photoplethysmography (PPG) sub-system configured to detect a PPG signal;
a response triggering module configured to analyze the PPG signal and output one or more response triggers based on a changing feature of the PPG signal within a time window, wherein each of the one or more response triggers relates to an instruction to initiate detection of at least one physiological characteristic of the individual; and
a blood pressure (BP) variability index determination module configured to determine a BP variability index related to the hemodynamic status of the individual based on a frequency or pattern of the one or more response triggers.

2. The system of claim 1, wherein the physiological characteristic of the individual includes a blood pressure of the individual.

3. The system of claim 1, wherein the BP variability index is based on the frequency of the one or more response triggers output by the response triggering module during the time window.

4. The system of claim 1, further comprising a BP detection unit operatively connected to the response trigger module and the BP variability index determination module, wherein the BP detection unit is configured to initiate detection of BP of the individual upon reception of the one or more response triggers.

5. The system of claim 4, wherein the BP variability index is further based on the detected BP of the individual.

6. The system of claim 1, wherein the system is devoid of a separate and distinct device configured to detect the at least one physiological characteristic of the individual.

7. The system of claim 1, further comprising a fluid responsiveness determination module in communication with the PPG sub-system and the BP variability index determination module, wherein the fluid responsiveness determination module is configured to determine a fluid responsiveness predictor (FRP) based on an analysis of the PPG signal.

8. The system of claim 7, wherein the BP variability index is further based on the FRP.

9. The system of claim 7, wherein the response triggering module is configured to output the one or more response triggers if the FRP is above or below a threshold or inside or outside of a range.

10. A system for determining a hemodynamic status of an individual comprising:
an input receiving a photoplethysmography (PPG) signal representing light absorption by a subject's tissue;
a trigger generated based on a change in a feature of the PPG signal, wherein the trigger indicates a probability of a change in blood pressure of the subject based on the change in the feature of the PPG signal; and
a calculator outputting a blood pressure (BP) variability index based on a number or pattern of triggers generated over a period of time.

11. The system of claim 10, further comprising a BP detection unit operatively connected to the calculator, wherein the BP detection unit is configured to initiate detection of BP of the individual upon reception of the trigger.

12. The system of claim 11, wherein the calculator outputs the BP variability index based on the detected BP of the individual.

13. The system of claim 10, wherein the system is devoid of a separate and distinct device configured to detect the BP of the individual.

14. The system of claim 10, further comprising a fluid responsiveness determination module in communication with the input, wherein the fluid responsiveness determination module is configured to determine a fluid responsiveness predictor (FRP) based on an analysis of the PPG signal.

15. The system of claim 14, wherein the BP variability index is further based on the FRP.

16. The system of claim 15, wherein the trigger is generated when the FRP is above or below a threshold or inside or outside of a range.

17. A system for determining a hemodynamic status of an individual, the system comprising:
at least one circuit or processor configured to determine whether to trigger a blood pressure (BP) measurement of the individual based on an analysis of a photoplethysmography (PPG) signal, and develop a BP variability index related to the hemodynamic status of the individual based on a number or pattern of triggered BP measurements within a time window.

18. The system of claim 17, wherein the system is devoid of a separate and distinct device configured to detect the BP of the individual.
